(19) United States
(12) Patent Application Publication
(10) Pub. No.: US 2025/0306143 A1
(43) Pub. Date: Oct. 2, 2025

(54) Ankle Coil

(71) Applicant: Siemens Healthineers AG, Forchheim (DE)

(72) Inventors: Bing Wu Zhang, Shenzhen (CN); Zeng He He, Shenzhen (CN); Tong Tong, Shenzhen (CN)

(73) Assignee: Siemens Healthineers AG, Forchheim (DE)

(21) Appl. No.: 19/092,005

(22) Filed: Mar. 27, 2025

(30) Foreign Application Priority Data
Mar. 27, 2024 (CN) 202420628225.0

(51) Int. Cl. G01R 33/34; A61B 5/00; A61B 5/055

(52) U.S. Cl. CPC G01R 33/34007; A61B 5/055; A61B 5/4595; A61B 5/702; G01R 33/34084

(57) ABSTRACT

An ankle coil may include a support assembly and a coil unit arranged inside the support assembly, which may include a seat including a side support located on a plantar side that supports a foot sole, and a bottom support located on a foot posterior side that supports a heel posterior part and a lower leg distal posterior part; flexible sheets connected to the seat and located on both sides of the foot, where the sheets are deformable to wrap the foot and overlap each other to provide an enclosing state; and a pressing member movably connected to the seat that may move until pressing against the overlapping portions in the enclosing state to maintain the enclosing state. Advantageously, the ankle coil is easy to operate, and the arrangement of the portion of the coil forming the coil unit in the pressing member helps to improve imaging quality.

### CROSS REFERENCE TO RELATED APPLICATIONS

This patent application claims priority to Chinese Patent Application No. 202420628225.0, filed Mar. 27, 2024, which is incorporated herein by reference in its entirety.

### BACKGROUND

### Technical Field

The present disclosure relates to the field of magnetic resonance imaging, in particular to an ankle coil.

### Related Art

Magnetic resonance imaging systems comprise an RF transmitting coil for generating a pulsed gradient magnetic field and an RF receiving coil for receiving nuclear magnetic resonance signals generated by atomic nuclei. The RF receiving coil can be either the transmitting coil itself or a separate coil that only receives RF signals.

An ankle coil is a magnetic resonance RF receiving coil designed for ankle scanning. At present, ankle coils usually comprise an upper part and a lower part that can be completely taken apart; before scanning, the lower half needs to first be put in place, then a subject's foot and finally the upper half. The upper half of the ankle coil is usually bulky and difficult to move and put in place, increasing the difficulty of operating the ankle coil.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 shows an ankle coil according to one or more exemplary embodiments of the disclosure.

FIG. 2 shows a varied state of the ankle coil shown in FIG. 1 according to one or more exemplary embodiments of the disclosure.

FIG. 3 shows a coil unit, of the ankle coil shown in FIG. 1, according to one or more exemplary embodiments of the disclosure.

FIG. 4 shows the overlapping of edges of flexible sheets according to one or more exemplary embodiments of the disclosure.

FIG. 5 shows a varied state of the ankle coil shown in FIG. 1 according to one or more exemplary embodiments of the disclosure.

FIG. 6 shows an ankle coil according to one or more exemplary embodiments of the disclosure.

FIG. 7 shows an ankle coil according to one or more exemplary embodiments of the disclosure.

FIG. 8 is a partial, sectionally view of a latch assembly according to one or more exemplary embodiments of the disclosure.

FIG. 9 shows an ankle coil according to one or more exemplary embodiments of the disclosure.

FIG. 10 shows an ankle coil according to one or more exemplary embodiments of the disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

An object of the present disclosure is to provide an ankle coil which is easy to operate.

The present disclosure provides an ankle coil, comprising a support assembly and a coil unit arranged inside the support assembly. The support assembly may comprise a seat, two flexible sheets and a pressing member. The seat may comprise a side support part that is located on a plantar side and is used for supporting a foot sole, and a bottom support part that is located on a foot posterior side and is used for supporting a heel posterior part and a lower leg distal posterior part. The two flexible sheets are connected to the seat and are configured to be able to be located on left and right sides of a foot, respectively. The two flexible sheets are deformable to wrap the foot and/or the lower leg distal end from an ankle anterior side and overlap each other, thereby achieving an enclosing state. The pressing member is movably connected to the seat. The pressing member is able to move until pressing against overlapping portions of the two flexible sheets in the enclosing state, to maintain the enclosing state of the two flexible sheets, and a portion of a coil forming the coil unit is provided in the pressing member.

When a subject's foot is to be placed on the ankle coil, the ankle coil does not need to be moved, which can facilitate operation. The arrangement of the portion of the coil forming the coil unit in the pressing member helps to improve imaging quality.

In another exemplary embodiment of the ankle coil, the pressing member has two connection ends connected to the seat. The two connection ends are rotatably connected to the side support part, which can improve the stability of the pressing member as a handle. The coil unit may comprise a first joining wire and a second joining wire. The first joining wire is located in the pressing member, and the second joining wire is located in the side support part. When the pressing member presses against the overlapping portions of the two flexible sheets in the enclosing state, the first joining wire and the second joining wire are in electrically conductive contact at the two connection ends to form an annular coil. This helps to improve imaging quality.

In another exemplary embodiment of the ankle coil, the pressing member is rotatably connected to the side support part, and the axis of rotation is parallel to the left-right direction of the human body. The pressing member has a pressing part at a rotating radial outer end thereof. During the rotation of the pressing member, the pressing part can bypass the tip of the foot. The pressing member is able to rotate to a pressing position. The pressing part of the pressing member in the pressing position is located on the dorsal side of the foot and is capable of pressing against the overlapping portions of the two flexible sheets in the enclosing state. Such a configuration can facilitate operation and helps to save space.

In another exemplary embodiment of the ankle coil, the support assembly may further comprise a strap. One end of the strap is fixedly connected to one of the flexible sheets. When the two flexible sheets are in the enclosing state, the other end of the strap can be detachably connected to the other flexible sheet, to maintain the enclosing state of the two flexible sheets. This helps to improve imaging quality.

In another exemplary embodiment of the ankle coil, the support assembly may further comprise a latch assembly. The latch assembly may comprise a first latch member and a second latch member. The first latch member is mounted to one of the flexible sheets. The second latch member is mounted to the other flexible sheet. When the two flexible sheets are in an enclosing state, the first latch member and the second latch member are able to form a snap-fit connection, to maintain the enclosing state of the two flexible sheets. This helps to further improve imaging quality.

In another exemplary embodiment of the ankle coil, the latch assembly may further comprise a first contact member arranged on the first latch member and a second contact member arranged on the second latch member. When the first latch member has formed a snap-fit connection with the second latch member, the first contact member is in electrically conductive contact with the second contact member. The coil unit may further comprise a third joining wire and a fourth joining wire. A third joining wire is arranged in the flexible sheet that is fitted with the first latch member and is electrically connected to the first contact member. A fourth joining wire is arranged in the flexible sheet that is fitted with the second latch member and is electrically connected to the second contact member. When the first latch member has formed a snap-fit connection with the second latch member, the third joining wire and the fourth joining wire conduct to form a complete coil. This can make the arrangement of the coil more flexible.

In another exemplary embodiment of the ankle coil, when the two flexible sheets are in the enclosing state, edges of the two flexible sheets overlap. Each flexible sheet is thinned at the edge to form a positioning recess. When the two flexible sheets are in the enclosing state, the edge of each flexible sheet is embedded in the positioning recess of the other flexible sheet. This allows the two flexible sheets to overlap each other in a standard overlapping range, which helps to improve the imaging quality.

In another exemplary embodiment of the ankle coil, a conducting wire of the coil unit is distributed in both mutually overlapping portions of the two flexible sheets in the enclosing state, to achieve overlapping decoupling of the coil. This helps to improve imaging quality.

In another exemplary embodiment of the ankle coil, the support assembly may further comprise a base pad. The base pad is connected to the bottom support part and forms a bottom recess having a rectangular U-shape cross-section. The bottom recess is used for accommodating posterior parts of a heel and a lower leg distal end. The base pad can limit left and right rocking of the subject's foot, thereby helping to improve imaging quality.

In another exemplary embodiment of the ankle coil, the support assembly may further comprise a rotating support frame. The seat is rotatably connected to the rotating support frame, and the axis of rotation is parallel to the left-right direction of the human body. This makes it easy to adjust the angle of the seat to suit different subjects.

As used herein, “schematic” means “serving as an instance, example or illustration”. No drawing or embodiment described herein as “schematic” should be interpreted as being a more preferred or more advantageous technical solution.

As used herein, “first”, “second”, “third”, “fourth”, etc. do not indicate order or degree of importance, etc., merely being used to indicate a distinction between parts, to facilitate description herein.

To make the drawings appear uncluttered, only those parts relevant to the present disclosure are shown schematically in the drawings; they do not represent the actual structure thereof as a product.

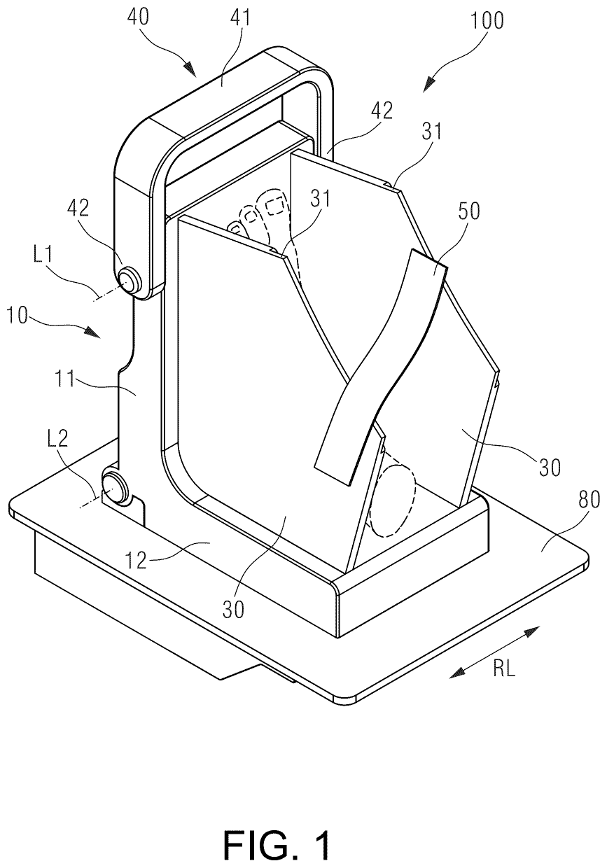

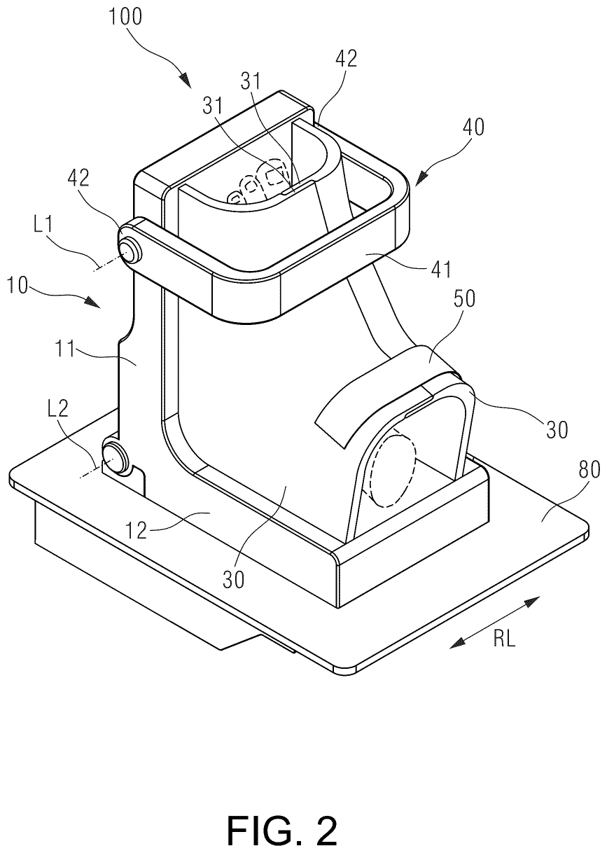

FIG. 1 is a structural schematic drawing of a exemplary embodiment of an ankle coil. FIG. 2 is a diagram of a varied state of the ankle coil shown in FIG. 1. As shown in FIGS. 1 and 2, the ankle coil may comprise a support assembly 100 and a coil unit arranged inside the support assembly 100 (the coil unit is not visible in FIGS. 1 and 2). For ease of understanding, the structure of the ankle coil is illustrated here with the aid of the left-right direction RL of the human body in anatomical terminology.

As shown in FIGS. 1 and 2, the support assembly 100 may comprise a seat 10, two flexible sheets 30 and a pressing member 40. The seat 10 may comprise a side support part 11 that is located on a plantar side and is used for supporting a foot sole, and a bottom support part 12 that is located on a foot posterior side and is used for supporting a heel posterior part and a lower leg distal posterior part. The two flexible sheets 30 are connected to the seat 10 and are configured to be able to be located on left and right sides of a foot, respectively, that is, exhibiting the state shown in FIG. 1. The two flexible sheets 30 can bend and deform into the state shown in FIG. 2 on the basis of the state shown in FIG. 1, to wrap the foot and the lower leg distal end from an ankle anterior side and overlap each other, thereby achieving an enclosing state (the state shown in FIG. 2 is the enclosing state). The housing 10 is made, for example, of a hard material to minimize its deformation during use. The flexible sheet 30 is made, for example, of a soft material to facilitate its switching between the state shown in FIG. 1 and the state shown in FIG. 2. In another exemplary embodiment, the two flexible sheets 30 may also be configured to wrap only one of the foot and the lower leg distal end from an ankle anterior side.

The pressing member 40 is movably connected to the seat 10. The pressing member 40 is able to move until pressing against overlapping portions of the two flexible sheets 30 in the enclosing state, to maintain the enclosing state of the two flexible sheets 30 during magnetic resonance imaging, thereby helping to improve imaging quality. Specifically, in the present exemplary embodiment, the pressing member 40 is rotatably connected to the seat 10 about a first axis L1. When the pressing member 40 is rotated to the position shown in FIG. 1, it does not affect the deformation of the flexible sheets 30 from the state shown in FIG. 1 to the state shown in FIG. 2, at which time the pressing member may be used, for example, as a handle to facilitate moving the ankle coil; when rotated to the pressing position shown in FIG. 2, the pressing member can press against the overlapping portions of the two flexible sheets 30 in the enclosing state, to maintain the enclosing state of the two flexible sheets 30 (that is, maintaining the state shown in FIG. 2). The means of rotational connection can facilitate operation, but the present disclosure is not limited to this; in another exemplary embodiment, the pressing member 40 may be movably connected to the seat 10 in another way.

A portion of a coil forming the coil unit 200 is provided in the pressing member 40; the shape of this portion of coil can be configured according to requirements, to help to improve imaging quality.

When a subject's foot is to be placed on the ankle coil, the ankle coil is adjusted to the state shown in FIG. 1, and then the subject's foot is placed on the seat 10; specifically, the subject's foot abuts the side support part 11, and the subject's heel posterior part and lower leg distal posterior part rest on the bottom support part 12. Next, the two flexible sheets 30 are then operated to deform them into the state shown in FIG. 2. Finally, the pressing member 40 is operated to move it to the pressing position shown in FIG. 2. The ankle coil does not need to be moved during the process, which can thereby facilitate operation.

As shown in FIGS. 1 and 2, in the present exemplary embodiment, the pressing member 40 is rotatably connected to the side support part 11 about a first axis L1, and the first axis L1 is parallel to the left-right direction RL of the human body. The pressing member 40 has a pressing part 41 at a rotating radial outer end thereof (i.e. the upper end in FIG. 1). During the rotation of the pressing member 40, the pressing part 41 can bypass the tip of the foot. The pressing part 41 of the pressing member 40 in the pressing position is located on the dorsal side of the foot and is capable of pressing against the overlapping portions of the two flexible sheets 30 in the enclosing state. Such a configuration can facilitate operation and helps to save space. When the pressing member 40 simultaneously acts as a handle, the pressing part 41 is for example a gripping portion of the handle, and the pressing part 41 can facilitate the pulling of the pressing member 40 as a handle.

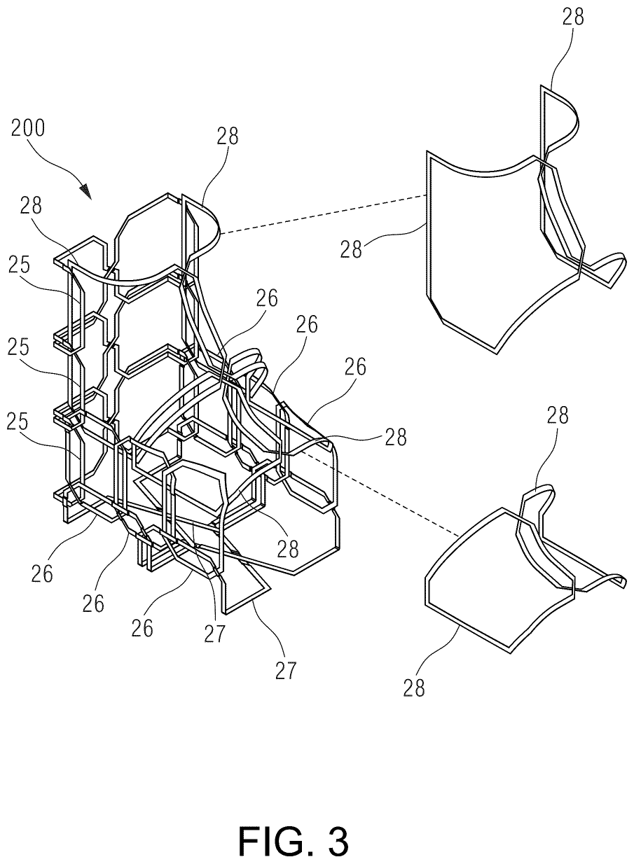

In an exemplary embodiment, a conducting wire of the coil unit 200 is distributed in both mutually overlapping portions of the two flexible sheets 30 in the enclosing state, to achieve overlapping decoupling of the coil. This helps to improve imaging quality. A specific description is given below. Since the coil unit of the ankle coil is completely disposed inside the support assembly 100, which is not visible in FIGS. 1 and 2, we show the coil unit 200 of the ankle coil separately in FIG. 3 (in which the portion of the coil which forms the coil unit 200 arranged in the pressing member 40 is not shown). The state of the coil unit 200 shown in FIG. 3 corresponds to the state of the ankle coil shown in FIG. 2. As shown in FIG. 3, the coil unit 200 may comprise three plantar coils 25 for covering the sole of the foot, six lateral coils 26 for covering the left and right sides of the heel and the lower leg distal end, two posterior coils 27 for covering the posterior parts of the heel and the lower leg distal end, and four anterior coils 28 for covering the anterior parts of the heel and the lower leg distal end. For ease of illustration, the four anterior coils 28 are shown separately on the right in FIG. 3, and the four anterior coils 28 are divided into two upper and lower groups as shown in FIG. 3, with the two anterior coils 28 in each group being located in two flexible sheets 30, respectively. When the two flexible sheets 30 are in the enclosing state, the two anterior coils 28 in each group overlap to decouple.

As shown in FIGS. 1 and 2, in an exemplary embodiment, the support assembly 100 may further comprise a strap 50. One end of the strap 50 is fixedly connected to one of the flexible sheets 30. When the two flexible sheets 30 are in the enclosing state, the other end of the strap 50 can be detachably connected to the other flexible sheet 30, to maintain the enclosing state of the two flexible sheets 30 (that is, maintaining the state shown in FIG. 2). This helps to improve imaging quality. In an exemplary embodiment, the other end of the strap 50 is detachably connected to the other flexible sheet 30, such as by a hook-and-loop fastener, which is inexpensive and convenient to operate, but the present disclosure is not limited to this.

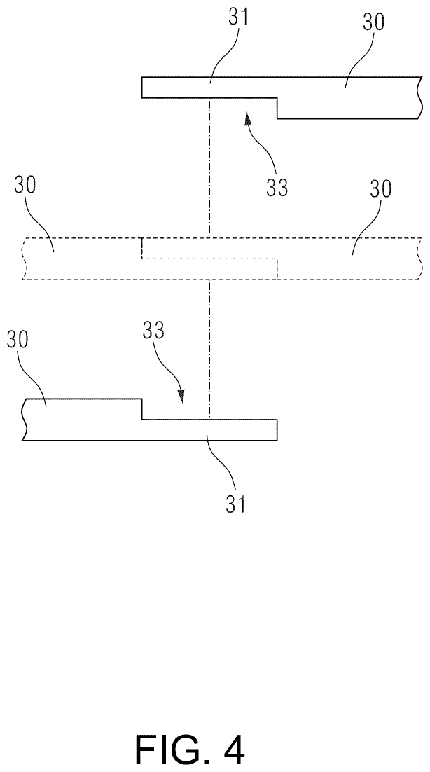

As shown in FIG. 2, in an exemplary embodiment, when the two flexible sheets 30 are in the enclosing state, edges 31 of the two flexible sheets 30 overlap. FIG. 4 shows a separated state of the two flexible sheets 30 and an overlapping state of the edges 31 in solid and dashed lines, respectively. As shown in FIG. 4, each flexible sheet 30 is thinned at the edge 31 to form a positioning recess 33. When the two flexible sheets 30 are in the enclosing state, the edge 31 of each flexible sheet 30 is embedded in the positioning recess 33 of the other flexible sheet 30. This allows the two flexible sheets to overlap each other in a standard overlapping range, which helps to improve the imaging quality.

As shown in FIGS. 1 and 2, in the present exemplary embodiment, the pressing member 40 is semi-annular and has two connection ends 42 connected to the seat 10. The two connection ends 42 are rotatably connected to the side support part 11. This can improve the stability of the pressing member as a handle.

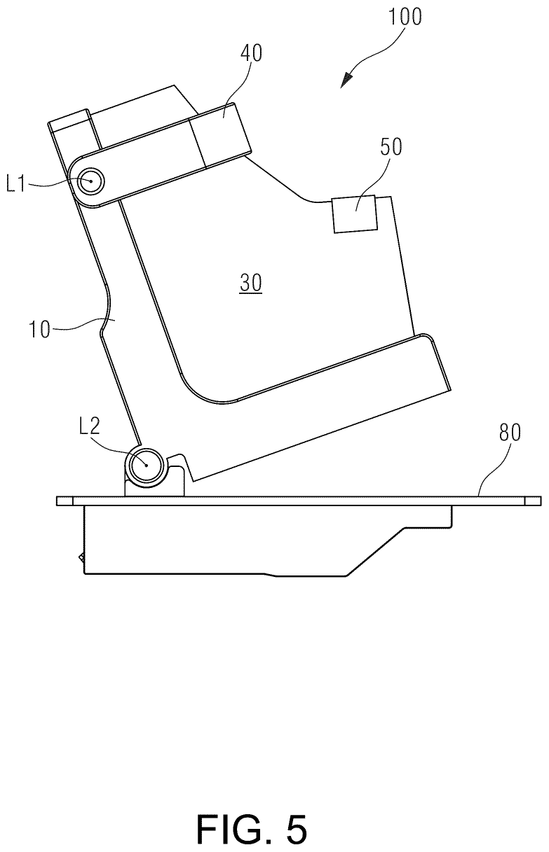

As shown in FIGS. 1 and 2, in an exemplary embodiment, the support assembly 100 may further comprise a rotating support frame 80. The seat 10 is rotatably connected to the rotating support frame 80 about a second axis L2, and the second axis L2 is parallel to the left-right direction RL of the human body. FIG. 5 shows a state of the seat 10 after an angle of rotation relative to the rotating support frame 80 on the basis of the state shown in FIG. 2. This makes it easy to adjust the angle of the seat to suit different subjects. A lower end of the rotating support frame 80, for example, can be connected to a medical table of a magnetic resonance imaging system by insertion, and the coil unit 200 is connected to a signal processing unit of the magnetic resonance imaging system, for example, by a joint arranged on the rotating support frame 80, but the present disclosure is not limited to this.

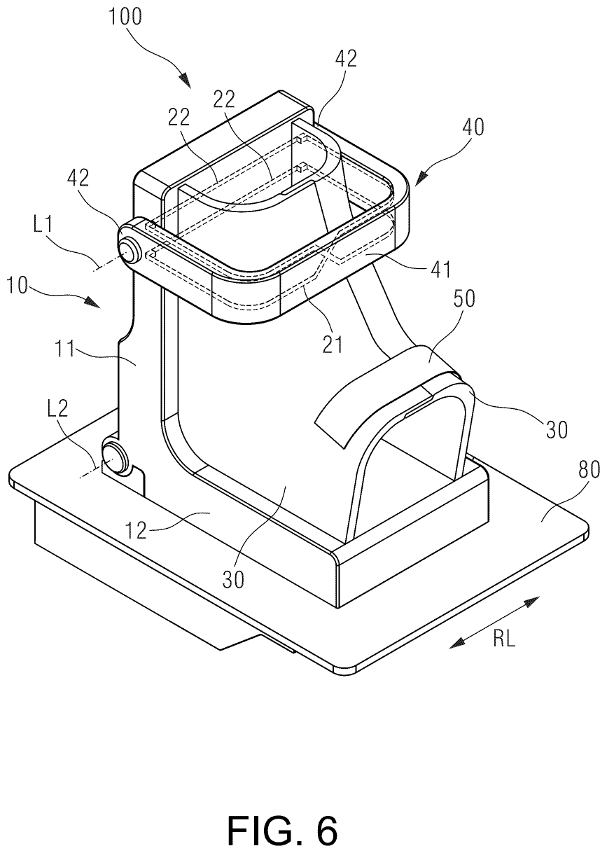

FIG. 6 is a structural schematic drawing of another exemplary embodiment of an ankle coil. Features of the ankle coil of the present exemplary embodiment which are identical or similar to those of the ankle coil shown in FIG. 1 will not be described again here; features which differ are as follows. As shown in FIG. 6, in the present exemplary embodiment, a portion of the coil forming the coil unit 200 that is arranged in the pressing member 40 is a first joining wire 21. The coil unit 200 may further comprise a second joining wire 22. The second joining wire 22 is located in the side support part 11. When the pressing member 40 is in a pressing position (the pressing member 40 in FIG. 6 being in the pressing position), the first joining wire 21 and the second joining wire 22 are in electrically conductive contact at the two connection ends 42 (for example by electrical contacts) to form an annular coil. The annular coil is, for example, a double-ring annular coil. This helps to improve imaging quality.

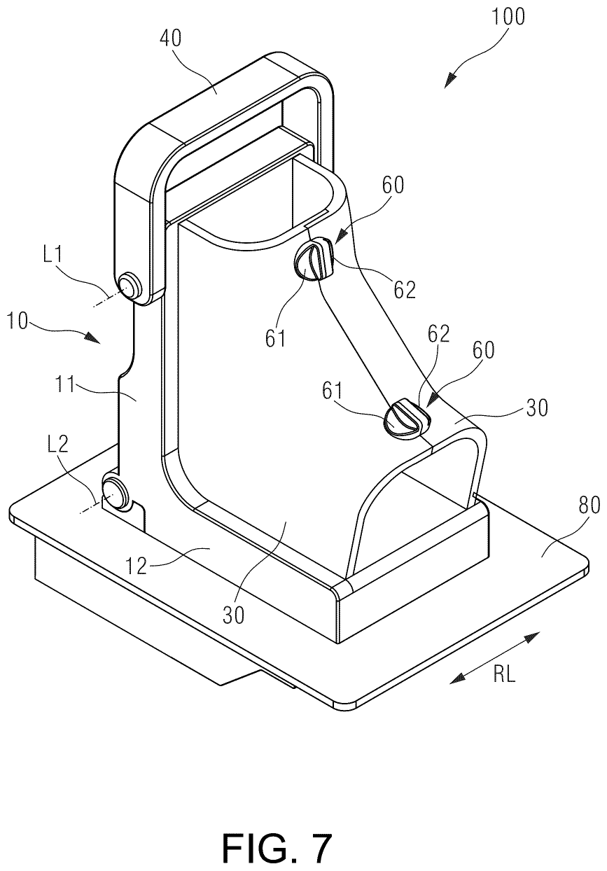

FIG. 7 is a structural schematic drawing of another exemplary embodiment of an ankle coil. Features of the ankle coil of the present exemplary embodiment which are identical or similar to those of the ankle coil shown in FIG. 1 will not be described again here; features which differ are as follows. As shown in FIG. 7, in the present exemplary embodiment, the support assembly 100 is not provided with a strap 50 but rather a latch assembly 60. Each latch assembly 60 may comprise a first latch member 61 and a second latch member 62. The first latch member 61 is mounted to one of the flexible sheets 30. The second latch member 62 is mounted to the other flexible sheet 30. When the two flexible sheets 30 are in an enclosing state, the first latch member 61 and the second latch member 62 are able to form a snap-fit connection, to maintain the enclosing state of the two flexible sheets 30. This helps to further improve imaging quality. In an exemplary embodiment, the number of latch assemblies 60 may be adjusted as required.

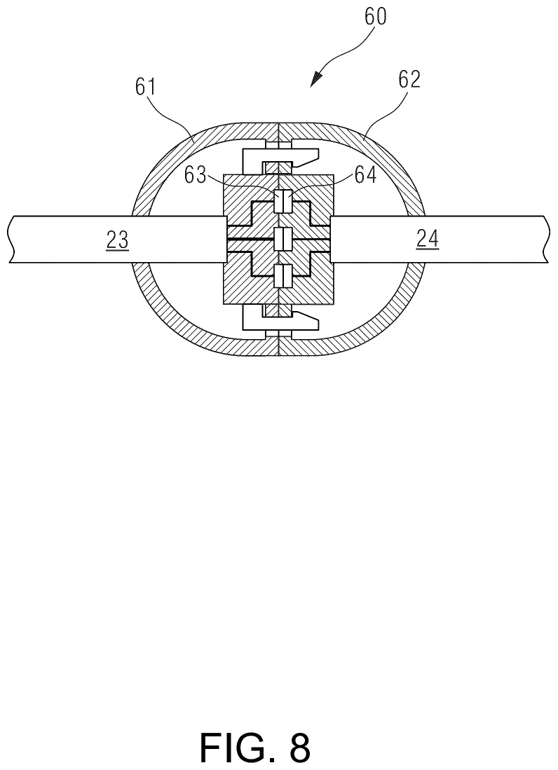

FIG. 8 is used to show a partial sectionally viewed structure of a latch assembly 60. As shown in FIG. 8, in an exemplary embodiment, the latch assembly 60 may further comprise a first contact member 63 arranged on the first latch member 61 and a second contact member 64 arranged on the second latch member 62. When the first latch member 61 has formed a snap-fit connection with the second latch member 62, the first contact member 63 is in electrically conductive contact with the second contact member 64. The coil unit 200 may further comprise a third joining wire 23 and a fourth joining wire 24. A third joining wire 23 is arranged in the flexible sheet 30 that is fitted with the first latch member 61 and is electrically connected to the first contact member 63. A fourth joining wire 24 is arranged in the flexible sheet 30 that is fitted with the second latch member 62 and is electrically connected to the second contact member 64. When the first latch member 61 has formed a snap-fit connection with the second latch member 62 (the first latch member 61 and the second latch member 62 in FIG. 8 forming a snap-fit connection), the third joining wire 23 and the fourth joining wire 24 conduct to form a complete coil. This can make the arrangement of the coil more flexible. It should be noted that, in this embodiment, it is also possible to provide only two latch members instead of the pressing member 40.

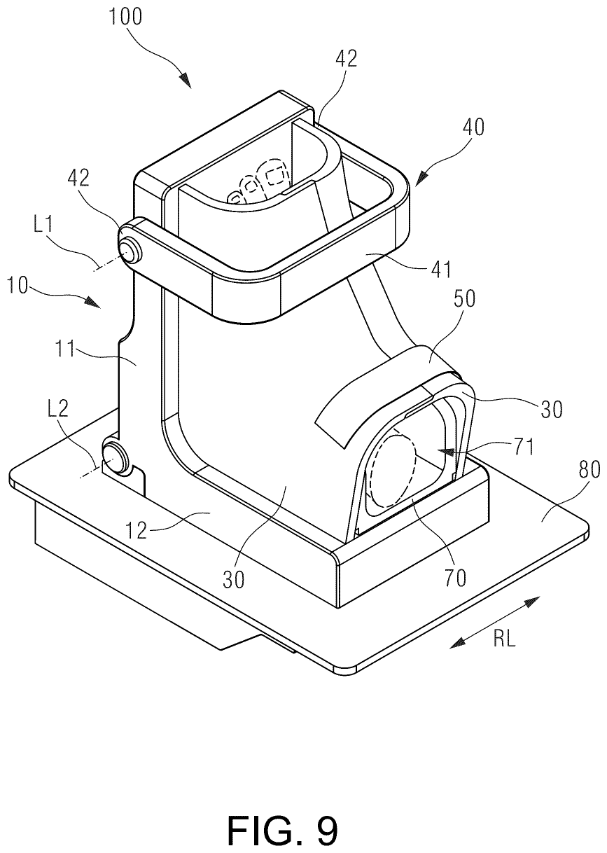

FIG. 9 is a structural schematic drawing of another exemplary embodiment of an ankle coil. Features of the ankle coil of the present exemplary embodiment which are identical or similar to those of the ankle coil shown in FIG. 1 will not be described again here; features which differ are as follows. As shown in FIG. 9, in the present exemplary embodiment, the support assembly 100 may further comprise a base pad 70. The base pad 70 is connected to the bottom support part 12 and forms a bottom recess 71 having a rectangular U-shape cross-section. The bottom recess 71 is used for accommodating posterior parts of a heel and a lower leg distal end. The base pad 70 can limit left and right rocking of the subject's foot, thereby helping to improve imaging quality. A surface layer of the base pad 70 may be, for example, constructed with sponge to enhance the comfort of the subject.

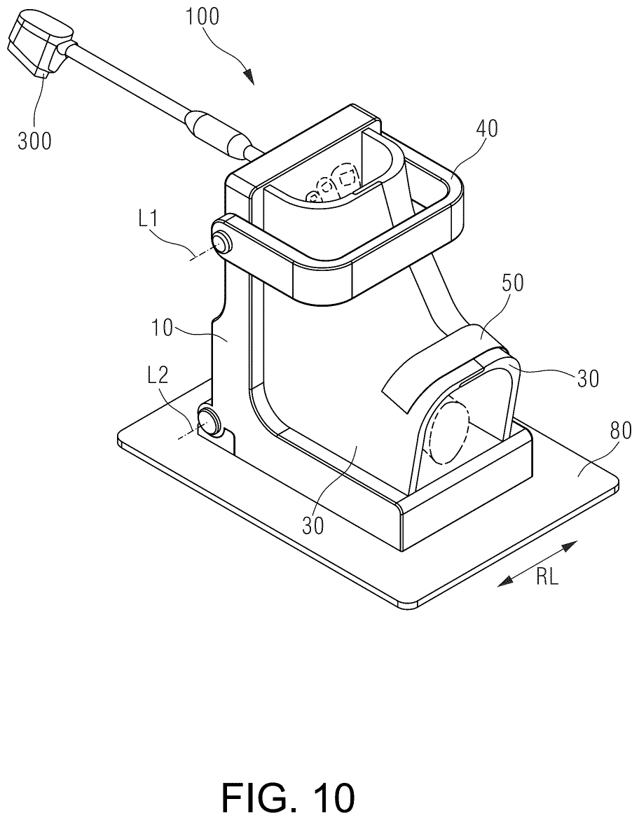

FIG. 10 is a structural schematic drawing of another exemplary embodiment of an ankle coil. Features of the ankle coil of the present exemplary embodiment which are identical or similar to those of the ankle coil shown in FIG. 1 will not be described again here; features which differ are as follows. As shown in FIG. 10, in the present exemplary embodiment, the ankle coil may further comprise a plug with cable 300, which is connected to the seat 10, and the coil unit 200 is connected to a signal processing unit of a magnetic resonance imaging system by means of the plug with cable 300. In the present exemplary embodiment, the rotating support frame 80 is placed directly on a medical table of the magnetic resonance imaging system, rather than connected to the medical table by insertion, and the rotating support frame 80 is also not provided with a joint connected to the coil unit 200. This type of ankle coil has better applicability.

It should be understood that although the description herein is based on various embodiments, it is by no means the case that each embodiment contains only one independent technical solution. Such a method of presentation is adopted herein purely for the sake of clarity. Those skilled in the art should consider the description in its entirety. The technical solutions in the various embodiments could also be suitably combined to form other embodiments understandable to those skilled in the art.

The series of detailed explanations set out above are merely particular explanations of feasible embodiments of the present disclosure, which are not intended to limit the scope of protection thereof. All equivalent embodiments or changes made without departing from the artistic spirit of the present disclosure, such as feature combinations, separations or repetitions, shall be included in the scope of protection thereof.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

### REFERENCE LIST

100 support assembly
10 seat
11 side support part
12 bottom support part
30 flexible sheet
31 edge
33 positioning recess
40 pressing member
41 pressing part
42 connecting end
50 strap
60 latch assembly
61 first latch member
62 second latch member
63 first contact member
64 second contact member
70 base pad
71 bottom slot
80 rotating support frame
200 coil unit
21 first joining wire
22 second joining wire
23 third joining wire
24 fourth joining wire
25 sole coil
26 side part coil
27 rear part coil
28 front part coil
300 plug with cable
RL left-right direction of human body
L1 first axis
L2 second axis

1. An ankle coil, comprising:
a support assembly; and
a coil unit arranged inside the support assembly, wherein the support assembly comprises:
a seat including a side support located on a plantar side and configured to support a foot sole, and a bottom support located on a foot posterior side and configured to support a heel posterior part and a lower leg distal posterior part;
two flexible sheets connected to the seat and configured to be located on left and right sides of a foot, respectively, the two flexible sheets being deformable to wrap the foot and/or the lower leg distal end from an ankle anterior side and overlap each other to provide an enclosing state; and
a pressing member movably connected to the seat, the pressing member being configured to move until pressing against overlapping portions of the two flexible sheets in the enclosing state to maintain the enclosing state of the two flexible sheets, wherein a portion of a coil forming the coil unit being provided in the pressing member.

2. The ankle coil according to claim 1, wherein:
the pressing member comprises two connection ends connected to the seat, the two connection ends being rotatably connected to the side support,
the coil unit comprises a first joining wire and a second joining wire, the first joining wire being located within the pressing member, the second joining wire being located within the side support, and
in response to the pressing member presses against the overlapping portions of the two flexible sheets in the enclosing state, the first joining wire and the second joining wire being in electrically conductive contact at the two connection ends to form an annular coil.

3. The ankle coil according to claim 1, wherein:
the pressing member is rotatably connected to the side support, an axis of rotation being parallel to a left-right direction of a human body, and
the pressing member comprises a pressing part at a rotating radial outer end thereof, the pressing part being configured to bypass a tip of the foot during rotation of the pressing member, the pressing member being configured to rotate to a pressing position, and the pressing part, in the pressing position, being located on a dorsal side of the foot and being configured to press against the overlapping portions of the two flexible sheets in the enclosing state.

4. The ankle coil according to claim 1, wherein the support assembly further comprises a strap, a first end of the strap being fixedly connected to one of the flexible sheets, and, in response to the two flexible sheets being in the enclosing state, a second end of the strap being detachably connected to the other flexible sheet, to maintain the enclosing state of the two flexible sheets.

5. The ankle coil according to claim 1, wherein the support assembly further comprises a latch assembly, the latch assembly including:
a first latch mounted to one of the flexible sheets; and
a second latch mounted to the other of the flexible sheets, wherein, in response to the two flexible sheets being in the enclosing state, the first latch and the second latch being configured to form a snap-fit connection to maintain the enclosing state of the two flexible sheets.

6. The ankle coil according to claim 5, wherein:
the latch assembly further comprises a first contact arranged on the first latch and a second contact arranged on the second latch,
in response to the first latch forming a snap-fit connection with the second latch, the first contact being in electrically conductive contact with the second contact,
the coil unit further comprises a third joining wire and a fourth joining wire, the third joining wire being arranged within one of the two flexible sheets fitted with the first latch and being electrically connected to the first contact, and the fourth joining wire being arranged within one of the two flexible sheets fitted with the second latch and being electrically connected to the second contact, and
in response to the first latch forming a snap-fit connection with the second latch, the third joining wire and the fourth joining wire conducting to form a coil.

7. The ankle coil according to claim 1, wherein:
in response to the two flexible sheets being in the enclosing state, respective edges of the two flexible sheets overlap,
each of the two flexible sheets being thinned at the respective edges to form a positioning recess, and,
in response the two flexible sheets being in the enclosing state, the respective edge of the flexible sheets is configured to be embedded in the positioning recess of the other of the two flexible sheets.

8. The ankle coil according to claim 1, wherein a conducting wire of the coil unit is distributed in both mutually overlapping portions of the two flexible sheets in the enclosing state to achieve overlapping decoupling of the coil.

9. The ankle coil according to claim 1, wherein the support assembly further comprises a base pad connected to the bottom support and forming a bottom recess having a rectangular U-shape cross-section, the bottom recess being configured to accommodate posterior parts of the heel and the lower leg distal end.

10. The ankle coil according to claim 1, wherein the support assembly further comprises a rotating support frame, the seat being rotatably connected to the rotating support frame, and an axis of rotation being parallel to a left-right direction of a human body.

11. A support assembly for an ankle coil, comprising:
a seat including a side support located on a plantar side and configured to support a foot sole, and a bottom support located on a foot posterior side and configured to support a heel posterior part and a lower leg distal posterior part;
two flexible sheets connected to the seat and configured to be located on left and right sides of a foot, respectively, the two flexible sheets being deformable to wrap the foot and/or the lower leg distal end from an ankle anterior side and overlap each other to provide an enclosing state; and
a pressing member movably connected to the seat, the pressing member being configured to move until pressing against overlapping portions of the two flexible sheets in the enclosing state to maintain the enclosing state of the two flexible sheets, wherein the pressing member being further configured to house at least a portion of a coil forming a coil unit.

12. The support assembly according to claim 11, wherein:
the pressing member comprises two connection ends connected to the seat, the two connection ends being rotatably connected to the side support,
the coil unit comprises a first joining wire and a second joining wire, the first joining wire being located within the pressing member, the second joining wire being located within the side support, and
in response to the pressing member presses against the overlapping portions of the two flexible sheets in the enclosing state, the first joining wire and the second joining wire being in electrically conductive contact at the two connection ends to form an annular coil.

13. The support assembly according to claim 11, wherein:
the pressing member is rotatably connected to the side support, an axis of rotation being parallel to a left-right direction of a human body, and
the pressing member comprises a pressing part at a rotating radial outer end thereof, the pressing part being configured to bypass a tip of the foot during rotation of the pressing member, the pressing member being configured to rotate to a pressing position, and the pressing part, in the pressing position, being located on a dorsal side of the foot and being configured to press against the overlapping portions of the two flexible sheets in the enclosing state.

14. The support assembly according to claim 11, wherein the support assembly further comprises a strap, a first end of the strap being fixedly connected to one of the flexible sheets, and, in response to the two flexible sheets being in the enclosing state, a second end of the strap being detachably connected to the other flexible sheet, to maintain the enclosing state of the two flexible sheets.

15. The support assembly according to claim 11, wherein the support assembly further comprises a latch assembly, the latch assembly including:
a first latch mounted to one of the flexible sheets; and
a second latch mounted to the other of the flexible sheets, wherein, in response to the two flexible sheets being in the enclosing state, the first latch and the second latch being configured to form a snap-fit connection to maintain the enclosing state of the two flexible sheets.

16. The support assembly according to claim 15, wherein:
the latch assembly further comprises a first contact arranged on the first latch and a second contact arranged on the second latch,
in response to the first latch forming a snap-fit connection with the second latch, the first contact being in electrically conductive contact with the second contact,
the coil unit further comprises a third joining wire and a fourth joining wire, the third joining wire being arranged within one of the two flexible sheets fitted with the first latch and being electrically connected to the first contact, and the fourth joining wire being arranged within one of the two flexible sheets fitted with the second latch and being electrically connected to the second contact, and
in response to the first latch forming a snap-fit connection with the second latch, the third joining wire and the fourth joining wire conducting to form a coil.

17. The support assembly according to claim 11, wherein:
in response to the two flexible sheets being in the enclosing state, respective edges of the two flexible sheets overlap,
each of the two flexible sheets being thinned at the respective edges to form a positioning recess, and,
in response the two flexible sheets being in the enclosing state, the respective edge of the flexible sheets is configured to be embedded in the positioning recess of the other of the two flexible sheets.

18. The support assembly according to claim 11, wherein a conducting wire of the coil unit is distributed in both mutually overlapping portions of the two flexible sheets in the enclosing state to achieve overlapping decoupling of the coil.

19. The support assembly according to claim 11, wherein the support assembly further comprises:
a base pad connected to the bottom support and forming a bottom recess having a rectangular U-shape cross-section, the bottom recess being configured to accommodate posterior parts of the heel and the lower leg distal end; and/or
a rotating support frame, the seat being rotatably connected to the rotating support frame, and an axis of rotation being parallel to a left-right direction of a human body.

20. An ankle coil comprising:
a support assembly according to claim 11; and
a coil unit arranged inside the support assembly.
